(19) United States
(12) Patent Application Publication
(10) Pub. No.: US 2004/0186545 A1
(43) Pub. Date: Sep. 23, 2004

(54) Temporary percutaneous cardioverter-defibrillator

(76) Inventors: Spencer Z. Rosero, Pittsford, NY (US); Stuart G. MacDonald, Pultneyville, NY (US); Jeffrey L. Helfer, Webster, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 10/392,506

(22) Filed: Mar. 20, 2003

(51) Int. Cl. A61N 1/05

(52) U.S. Cl. 607/119; 607/5

(57) ABSTRACT

An assembly that contains a device for the insertion of a temporary percutaneous cardioverter-defibrillator in contact with biological tissue of a biological organism. The assembly contains a conductor located on the periphery of the distal section of the assembly. The assembly is located in the vascular system of the biological organism, near the heart; it also contains a device for flowing electrical current through the conductor; and it also contains a cutaneous or subcutaneous electrode.

### FIELD OF THE INVENTION

A temporary percutaneous cardioverter-defibrillator comprised of a sheath on which a conductor is disposed, and a device for insertion of the same.

### BACKGROUND OF THE INVENTION

As is disclosed in published United States patent application 2002/0107544, defibrillation/cardioversion is a technique employed to counter arrhythmic heart conditions, such as some tachycardias in the atria and/or the ventricles. Generally electrodes are employed to stimulate the heart with electrical impulses or shocks which typically are of a magnitude greater than the pulses used in cardiac pacing. The entire disclosure of this United States patent application is hereby incorporated by reference into this specification.

Defibrillation/cardioversion is but one of the techniques used to care for critically ill patients. The acute and emergency care of critically ill patients often requires cardiac pacing support, cardioversion of heart rhythm, or elimination of heart fibrillation through defibrillation. Pacing support is typically provided by placing pacing leads into the heart, which are connected to a proximally located pulse generator. Cardioversion and/or defibrillation support is typically instituted using externally applied cardioversion-defibrillation (i.e. cardioverters-defibrillator “paddles”) or implanted cardioverter-defibrillators (i.e., ICDs). However, these procedures have significant limitations. For a discussion of defibrillation and cardioversion, reference may be had, e.g., to U.S. Pat. No. 6,349,233 (neuro-stimulation to control pain during cardioversion defibrillation), U.S. Pat. No. 6,067,471 (atrial and ventricular implantable cardioverter-defibrillator and lead system), U.S. Pat. Nos. 5,987,714, 5,836,942, 5,772,690 (surrogate defibrillation electrode for testing implantable cardioverter-defibrillators), U.S. Pat. No. 5,713,944 (cardioversion-defibrillation catheter lead having selectively exposable outer conductors), U.S. Pat. Nos. 5,476,497, 5,449,381 (endocardial catheter for defibrillation, cardioversion,and pacing), U.S. Pat. No. 5,439,481 (semi-automatic atrial and ventricular cardioverter defibrillator), U.S. Pat. No. 5,411,547 (implantable cardioversion-defibrillation patch), U.S. Pat. Nos. 5,405,362, 4,980,379, 4,320,763, and the like. The entire disclosure of each of these United States Patents is hereby incorporated by reference into this specification.

Externally applied cardioverter-defibrillator paddles require manual application in response to an electrophysiological signal, such as the absence of normal heart rhythm. The time required for personnel to recognize and react to such a signal introduces a significant delay in delivering the cardioversion-defibrillation therapy, often as much as two to five minutes, which places the patient at greater risk of injury or death. See, e.g., U.S. Pat. No. 5,148,805 (defibrillator pad system), U.S. Pat. No. 5,203,347(uni-cable defibrillator paddles), U.S. Pat. No. 5,123,423 (defibrillator pad assembly), U.S. Pat. Nos. 5,076,286, 4,998,536, 4,058,127, 4,002,239, and the like. The entire disclosure of each of these United States patents is hereby incorporated by reference into this specification.

Implanted cardioverter-defibrillators eliminate this delay in response time by sensing electrophysiological signals and automatically delivering cardioversion-defibrillation therapy. However, these devices are expensive and difficult to install and, thus, are not widely utilized in the acute and emergency are environment.

Furthermore, the very high levels of energy that are applied by external cardioverters-defibrillators can place the patient at great risk by damaging the internal cardiac pacemaker/

Published United States patent application 2002/0198583 describes and claims, in one embodiment, a system providing cardiac stimulation, comprising: a probe insertable through a mouth into an esophagus of a patient; a first group of conductors; a second group of conductors; and a disposable sheath slidably covering the probe and comprising the first group of conductors and the second group of conductors integrated therein providing a path of least resistance between one of the conductors in the first group of conductors and one of the conductors in the second group of conductors, wherein the first and the second groups of conductors are connected to a cardiac resuscitation apparatus via a single cable to provide the cardiac stimulation to the patient.

The disadvantage of prior art systems, such as those disclosed in United States patent application 2002/0198583, is they are cumbersome and time-consuming to use. It is an object of this invention to provide a device that overcomes the shortcomings of the prior art devices.

### SUMMARY OF THE INVENTION

In accordance with this invention, there is provided an assembly that contains apparatus for the insertion of a temporary percutaneous cardioverter-defibrillator in contact with biological tissue of a biological organism. The assembly contains a conductor located on the periphery of the distal section of the assembly, the assembly being located in the vascular system of the organism in near the heart; it also contains a device for flowing electrical current through the conductor, and a cutaneous or subcutaneous electrode.

### BRIEF DESCRIPTION OF THE DRAWINGS

The inventions will be described by reference to the following drawings, in which like elements refer to like numerals, and in which:

FIG. 1 is a schematic illustration of one apparatus of the present invention disposed within a human body;

FIG. 2 is a schematic sectional view of one preferred combined pacing and cardioverter-defibrillator lead

FIG. 3 is a sectional schematic view of a preferred embodiment of a combined introducer and cardioverter-defibrillator lead;

FIG. 4 is a schematic of an adapter for a pulse generator;

FIG. 5 is schematic of an integrity checking device;

FIG. 6 is a schematic illustration of another device of the invention; and

FIG. 7 is a schematic illustration of yet another device of the invention that contains a “tear-away” sheath.

### DESCRIPTION OF THE PREFERRED EMBODIMENT

The present invention, in one embodiment, provides a temporary implanted pacing and cardioverter-defibrillator that provides the advantages of prior art implanted cardioverters-defibrillators (e.g. rapid response time) but without the disadvantages of high cost, difficult installation, and potential to damage the pacing device. Other features and capabilities are provided which uniquely address the acute and emergency care needs of critically ill patients.

In one embodiment of the invention, there is provided a device and method for providing temporary pacing and cardioversion-defibrillation therapy. This device preferably is comprised of the one or more of the following subsystems: (1) an external and reusable pulse generator, (2) a combined distal pacing and cardioverter-defibrillator lead, (3) a combined introducer and proximal cardioverter-defibrillator lead, and (4) an external electrode.

In this embodiment, the proximal cardioverter-defibrillator lead is positioned below the surface of the skin at the point of introduction. The distal pacing and cardioverter-defibrillator lead is advanced through the introducer and positioned within the heart. The external pulse generator is coupled to both leads as well as to an external electrode that is attached to the skin of the patient, thereby forming a conductive path from the heart to cardioverters-defibrillator leads and external electrodes, thereby forming the necessary electrical paths for effective cardioversion-defibrillation. The external pulse generator then provides the required pacing support (e.g. VVO or VVI) as well as continuous monitoring of cardiac rhythm to determine when cardioversion-defibrillation pulses need to be applied.

This system provides the advantage of a rapid response time. The implanted leads enable continuous monitoring of heart rhythm and for determining when cardioversion-defibrillation pulses are required. Once this need is determined, the pulses can be applied automatically without the need for additional human intervention. Furthermore, the external pulse generator simplifies communication of sensory information, such as the onset or irregular cardiac rhythm and/or fibrillation to medical support staff, who can then either activate cardioversion-defibrillation remotely or more effectively administer follow-on treatments.

Another advantage of this system is ease of installation. In one embodiment, the present invention requires the implantation of a single pacing lead through an introducer, and the attachment of a third electrode to the surface of the skin—procedures that medical professionals are typically quite comfortable with. The more difficult need to implant a pulse generator is not required.

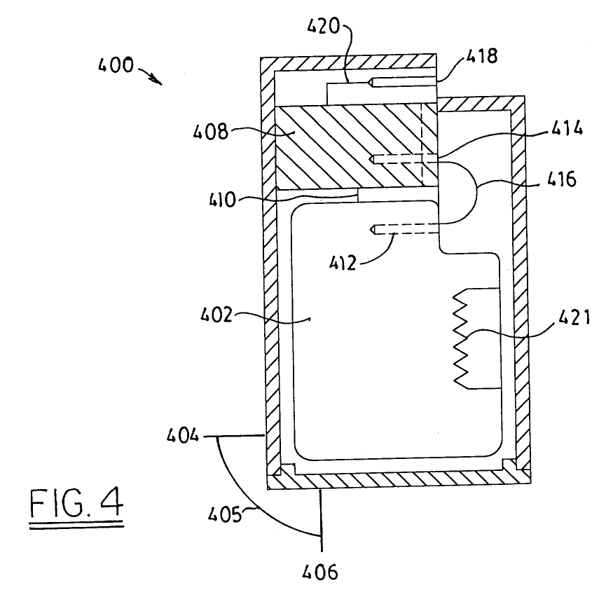

Another advantage of the system of this invention is reduced cost. The cost of the temporary pacing and cardioversion-defibrillation system is minimized by re-using an external pacemaker, which typically costs approximately $30,000. This re-use is enabled by the use of an adapter (see FIG. 4) which adapts pulse generator functions to the external placement of the pulse generator. The lead designs also enable a lower unit manufacturing cost and therefore a lower sales price.

Yet another advantage of the system of this invention is its flexibility. The proximal ends of the combined distal pacing/cardioverter-defibrillator lead and combined introducer and proximal cardioverter-defibrillator lead can be interfaced to existing electrical, fluidic, and other devices, which enable the leads to perform additional functions, such as delivery of fluids, pressure monitoring, and the like.

The optional tear-away introducer and proximal cardioverter-defibrillator lead also is adapted to be removed without disturbing the position or interrupting the operation of the distal pacing and cardioverter-defibrillator lead.

This feature is particularly important when continuous pacing is required, such as with pacemaker dependent patients.

Yet another advantage of the system of this invention is enhanced safety. The optional tear-away cardioversion-defibrillation lead enables this typically temporary lead to be removed without disrupting the position of the typically permanently implanted pacing lead. This reduces the risk of perforating thin blood vessels, dislodging loosely attached plaques, or creating injury to the surface of blood vessels which could cause injury that could result in stroke or longer term closure of the vessels.

Yet another advantage of the system of this invention is the improved sensitivity and specificity afforded by the system for the diagnosis and detection of arrhythmias as well as the ability to more effectively pace and/or defibrillate the heart.

Yet another advantage of the system of the invention is that it provides means to deliver cardioversion-defibrillation pulses directly to the heart where they can more effectively cardiovert or defibrillate heart tissues, as opposed to introducing such energy only through external electrodes, which introduce greater electrical impedance between the electrode and the tissues to be paced, cardioverted, or defibrillated, or all of these.

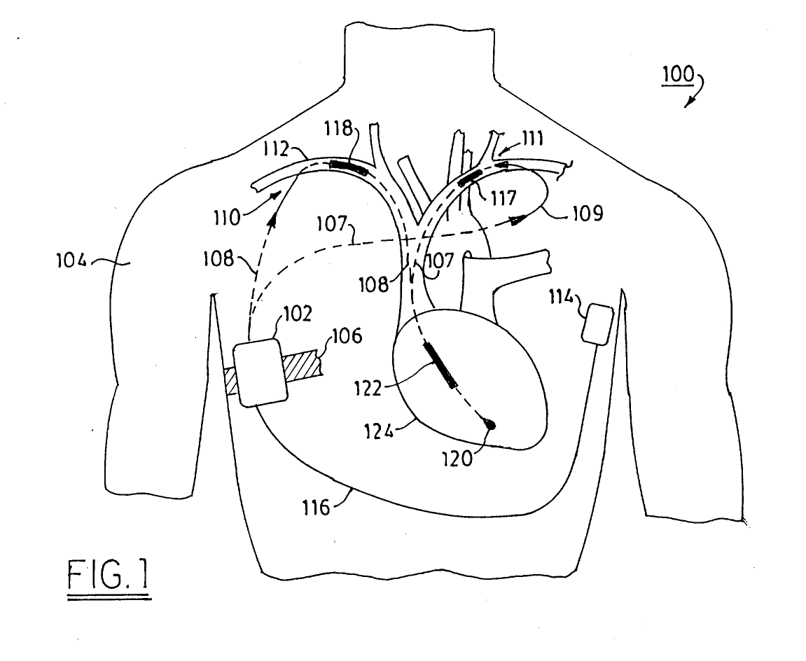

FIG. 1 is a schematic illustration of one preferred embodiment of this invention. Referring to FIG. 1, and in the preferred embodiment depicted therein, a temporary percutaneous cadioverter defibrillator assembly 100 is shown. In the preferred embodiment depicted in FIG. 1, assembly 100 is in contact with biological tissue.

Thus, and referring again to FIG. 1, it will be seen that system 100 is preferably is comprised of pulse generator 102. In the embodiment depicted, the pulse generator 102 is located outside of the biological organism 104 and, in one aspect of this embodiment, is attached to a patient 104 by means of attachment means 106.

One may use many of the pulse generators known to those skilled in the art. Alternatively, one may use many of the devices that comprise such pulse generators.

In one embodiment, the pulse generator 102 is a part of a defibrillation system. In another embodiment, the pulse generator 102 is a part of pacemaker system. In yet another embodiment, the pulse generator 102 is part of a cardioversion/anti-tachycardia pacing system.

In one embodiment, the pulse generator 102 is part of a single chamber or dual chamber defibrillation system. These systems are well known to those skilled in the art. Reference may be had, e.g., to U.S. Pat. No. 6,370,427 (dual chamber bi-ventricular pacing and defibrillation), U.S. Pat. Nos. 5,209,229, 6,327,499, 6,076,014, 5,476,502, 5,374,287, 6,134,470, 6,351,669, 6,501,988, 6,285,907, 5,456,706, 6,411,848, 6,353,759, 6,085,119, 5,193,350, 6,430,438, 5,683,447, and the like. The entire disclosure of these United States patents is hereby incorporated by reference into this specification.

The pulse generator 102 need not be contained in another system.

Referring again to FIG. 1, and in the preferred embodiment depicted therein, it will sent hat means 106 are provided to attach the pulse generator 102 either to the patient 104 and/or to a support near the patient 104. In one embodiment, means 106 is a holster (not shown) attached to the patient or a rail near the patient.

As will be apparent to those skilled in the art, FIG. 1 depicts two alternative means of inserting a catheter, one being the left subclavian vein approach, and the other being the right subclavian vein approach. These approaches are well known to those skilled in the art; reference may be had, e.g. to U.S. Pat. Nos. 6,530,876, 6,501,983 (implantable myocardial, ischemia detector), U.S. Pat. Nos. 6,328,699, 6,299,575, 6,136,025, 5,716,318, 5,678,570, 5,437,633, 5,433,730 (conductive pouch electrode for defibrillation), U.S. Pat. Nos. 5,423,334, 5,411,527 (defibrillation electrodes and implantation),U.S. Pat. Nos. 5,216,032, 4,946,457 (defibrillator system with cardiac leads), U.S. Pat. No. 4,884,567, and the like; and the entire disclosure of each of these United States patents is hereby incorporated by reference into this specification. In one embodiment, it is preferred to use the left subclavian vein approach in most cases.

Referring again to FIG. 1, and in the preferred embodiment depicted therein, it will be seen that attached to pulse generator 102 is a distal pacing and cardioverter-defibrillator catheter 107/108. The catheter 107 may be used for the left subclavian approach, and the catheter 108 may be used for the right subclavian approach. In either case, the catheter 107/108 passes through defibrillator sheath 109/110, depending upon whether the left or right subclavian approach is used.

In one aspect of the embodiment depicted, catheter 109/110 enters the patient 104 at veins 111/112 and is positioned in heart 124, In these aspects, an external surface electrode 114 is connected to the pulse generator via electrically conductive cable 116.

Referring again to FIG. 1, it will be seen that, attached to sheath assembly 109/110 and catheter 107/108 is proximal cardioversion-defibrillation electrode 118. In one embodiment, such proximal cardioversion-defibrillation electrode 118 is integrally connected to and part of the sheath assembly 109/110.

Referring again to FIG. 1, and in the preferred embodiment depicted therein, it will be seen that attached to catheter 107/108 is one or more pacing electrode(s) 120 and distal cardioversion-defibrillation electrodes 122 which are positioned in heart 124.

In one embodiment, where two or more pacing electrode 120 are used, the apparatus 100 is adapted to provide alternative means for cardiac stimulation using a bipolar electrode configuration within the catheter, or a unipolar configuration between the catheter and the other components of the system, such as proximal electrode 117/118 or external electrode 114.

In one embodiment, not shown in FIG. 1, the catheter 107/108 is optionally split into two segments, a distal segment and a proximal segment, such that the proximal segment can be removed and the internal segment attached to an implantable pulse generator (e.g. implantable pacemaker and/or cardioverters-defibrillator) so as to allow installation of a permanently implantable pacemaker and/or cardioverters-defibrillator without the need to remove distal pacing and catheter 107/108. In this aspect, sheath assembly 109/110 and catheter 107/108 is preferably removed by either sliding sheath 109/110 over catheter 107/108(which requires momentary detatachment of catheter 107/108 from pulse generator 102), or preferably by “tearing away” sheath assembly 109/110.

Referring again to FIG. 1, and once the pulse generator 102 has been installed as illustrated, in one embodiment the pulse generator 102 monitors heart rhythm and determines automatically when pacing and/or cardioversion-defibrillation is required. Alternately, and in another embodiment, pulse generator 102 monitors heart rhythm and notifies medical support staff when pacing and/or cardioversion-defibrillation is required, which staff then enable the pulse generator 102 to administer this therapy.

Referring again to FIG. 1, it will be apparent that portions of the assembly 100 are in contact with biological tissue. Thus, e.g., defibrillation electrode 122 is in contact with cardiac tissue. Thus, e.g., defibrillation electrode 118 is in contact with blood.

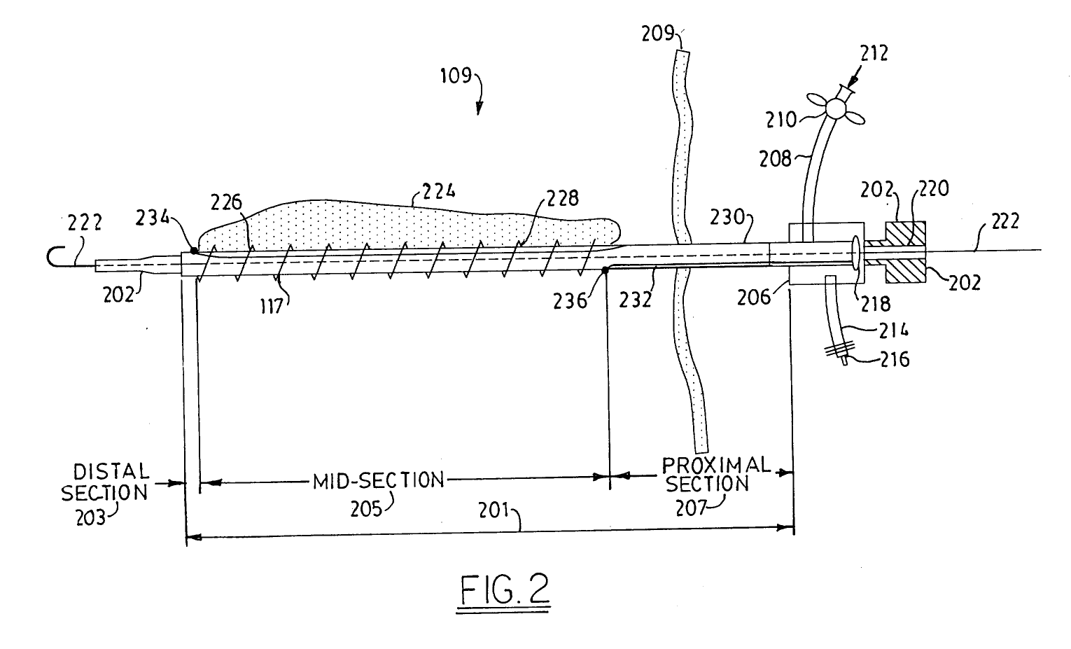

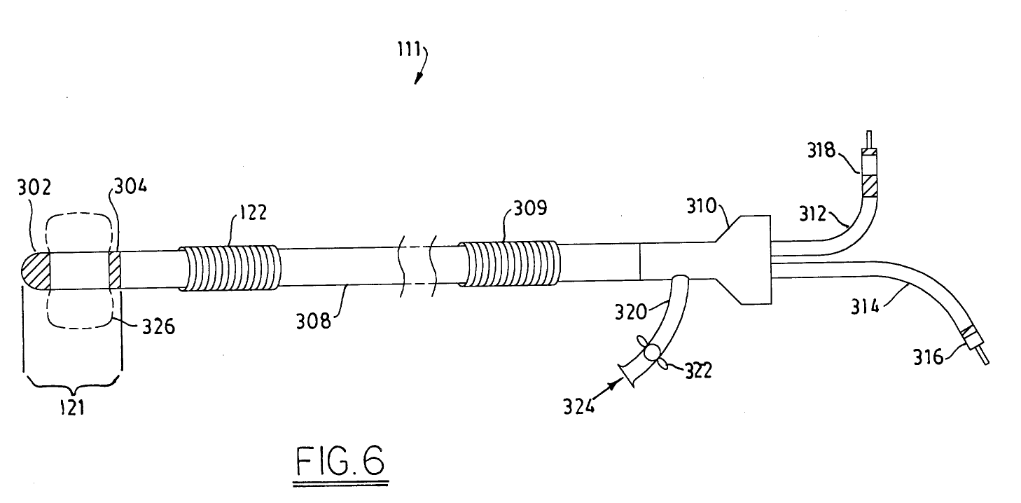

In another embodiment of the invention, and referring again to FIG. 1, the sheath 109 (see FIG. 2) is omitted. Reference also may be had to FIG. 6 for another embodiment of the invention.

FIG. 2 is a schematic illustration of a preferred embodiment of sheath assembly 109. It will be seen that sheath assembly 109 is comprised an introducer 202. through which a guide wire 222 is disposed, and a cardioversion-defibrillation sheath electrode 118 integrally connected to said introducer 202; the sheath electrode 118, in the embodiment depicted, is integrally connected attached to block 206.

The block 206 preferably is a manifold adapted to allow for the connection into the patient 104 (see FIG. 1) of various electrical currents (not shown), and/or guide wire 222, etc., and/or therapeutic agent(s), into the vascular space (not shown in FIG. 2).

The block 206, in the embodiment depicted, is attached to lumen 208, stopcock 210, port 212, lumen 214, and pulse generator connector 216, and also to diaphragm valve 218. This block assembly, and/or comparable block assemblies, are is commercially available (as, e.g., defibrillation electrode [DF-1] connector).

Referring again to FIG. 2, and in the embodiment depicted, introducer 202 preferably contains a hollow lumen 220 which enables the introducer to pass over guide wire 222. Cardioversion-defibrillation electrode 204 is preferably electrically attached, by means not shown, to pulse generator connector 216.

In the embodiment depicted in FIG. 2, a conductor 117 is disposed over the middle section 205 of the assembly 109. In the embodiment depicted, the sheath assembly 109 is preferably comprised of a distal section 203, a mid section 205, and proximal section 207; these sections are preferably integrally connected to each other.

Referring again to FIG. 2, and in one aspect of this embodiment, at least one centimeter of the distal section 203 is not contiguous with the conductor 117. In another aspect, at least about 2 centimeters of the proximal section 207 is not contiguous with the conductor 117.

The conductor 117 preferably is comprised of or consists essentially of conductive material such as stainless steel, titanium, nitinol, conductive polymeric material, conductive ceramic material, etc. In one embodiment, the conductor 117 is comprised or consists essentially of biocompatible material. It is preferred that the conductivity of the conductor 117 be relatively high.

In one embodiment, depicted in FIG. 2, the conductor 117 is in the shape of a coil, and the coil 117 is preferably in contact with tissue/blood 224. In one aspect of this embodiment, the conductor 117 presents a surface area to the tissue 224 of from about 300 to about 800 square millimeters and, more preferably, from about 400 to about 700 square millimeters. The surface area presented to tissue 224 is referred to hereinafter as the effective surface area of the conductor; and it will vary with the conductor size and length.

In one embodiment, and referring again to FIG. 2, the current density at the proximal end 226 of the coil 117 is no more than about 30 percent different than the current density at the distal end 228 of the coil 117. One may use conventional means to insure that the difference in such current densities is minimal. In one aspect of this embodiment, the conductor 230 is connected in parallel with conductor 232 at points 234 and 236. Other means of minimizing the difference in such current densities also may be used.

In one embodiment, the current densities on and/or in conductor 117 do not exceed about 30 watts per square millimeter.

In one embodiment, the current flow through conductor 117 is regulated such that the temperature of tissue 224 does not exceed about 41 degrees Celsius.

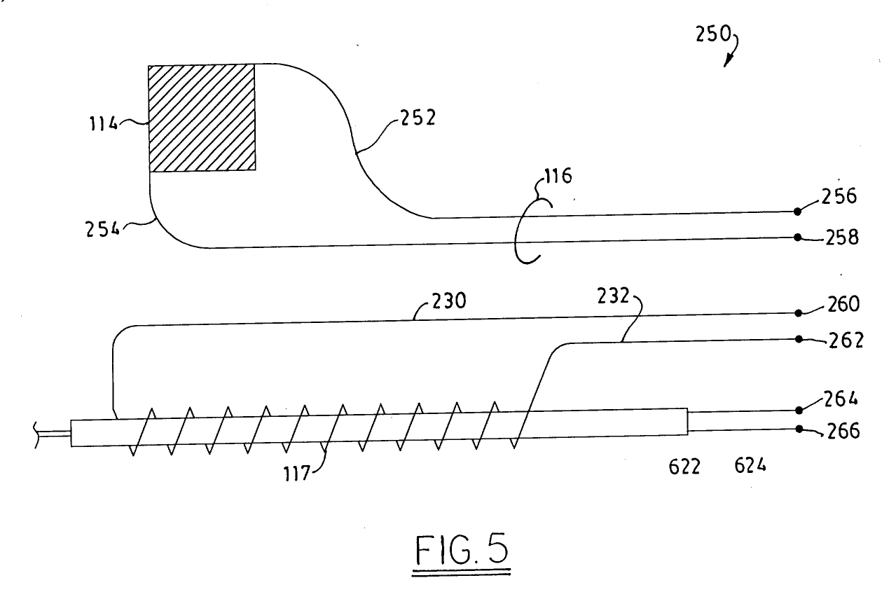

In one embodiment, the assembly 109 provides a means for checking the integrity of the conductor 204. One such means is depicted in FIG. 5.

One preferred device 250 for checking the integrity of the conductor 204 is comprised of patch electrode 114 (see FIG. 1) attached to conductor assembly 116. In the embodiment depicted, conductor assembly 116 is comprised of conductor 252, conductor 254, and switch contact points 256 and 258. When the integrity of the circuit is to be checked, contacts 256 and 258 are electrically separated. An electrical circuit (not shown) is adapted to make and/or break contact between contact points 256, 258, 260, 262, 264, and 266.

Referring again to FIG. 5, the conductor 117 is connected to contact points 260 and 262 by means of conductors 230 and 232 (see FIG. 2).

The pulse generator 102 (not shown in FIG. 5) can periodically make or break connections with contact points 256, 258, 260, 262, 264, and/or 266 to check the integrity of the circuits. Alternatively, one may use other circuit integrity testing means.

By way of illustration and not limitation, devices for checking the integrity of electrical circuits are described in U.S. Pat. No. 6,433,572 (integrated circuit integrity analysis), U.S. Pat. No. 6,424,136 (fault assessments of loop circuit integrity), U.S. Pat. Nos. 6,395,815, 6,055,981, 6,032,187, 5,552,712 (in-place circuit integrity testing), U.S. Pat. No. 5,532,601 (circuit integrity test system), U.S. Pat. Nos. 5,381,438, 5,173,960, 5,138,266, 5,055,774 (integrated circuit integrity testing apparatus), U.S. Pat. No. 4,639,719(apparatus for monitoring circuit integrity), U.S. Pat. No. 4,165,270 (circuit integrity tester), and the like. The entire disclosure of each of these United States patents is hereby incorporated by reference into this specification.

Referring again to FIG. 2, and in one embodiment thereof, when conductor 117 is in the shape of a coil, the conductor 117 preferably is flexible.

One may use conductors 117 that are either not in the shape of a coil and/or are not comprised of metallic conductive material. Thus, by way of illustration and not limitation, conductor 117 may be comprised of a conductive polymer, and/or a conductive ceramic material, and/or a conductive foam, etc.

In one embodiment, the conductor 204 is comprised of coated with antithrombogenic material.

Antithrombogenic compositions and structures have been well known to those skilled in the art for many years. As is disclosed, e.g., in U.S. Pat. No. 5,783,570, the entire disclosure of which is hereby incorporated by reference into this specification, “Artificial materials superior in processability, elasticity and flexibility have been widely used as medical materials in recent years. It is expected that they will be increasingly used in a wider area as artificial organs such as artificial kidney, artificial lung, extracorporeal circulation devices and artificial blood vessels, as well as disposable products such as syringes, blood bags, cardiac catheters and the like. These medical materials are required to have, in addition to sufficient mechanical strength and durability, biological safety which particularly means the absence of blood coagulation upon contact with blood, i.e., antithrombogenicity.”

“Conventionally employed methods for imparting antithrombogenicity to medical materials are generally classified into three groups of (1) immobilizing a mucopolysaccharide (e.g., heparin) or a plasminogen activator (e.g., urokinase) on the surface of a material, (2) modifying the surface of a material so that it carries negative charge or hydrophilicity, and (3) inactivating the surface of a material. Of these, the method of (1) (hereinafter to be referred to briefly as surface heparin method) is further subdivided into the methods of (A) blending of a polymer and an organic solvent-soluble heparin, (B) coating of the material surface with an organic solvent-soluble heparin, (C) ionical bonding of heparin to a cationic group in the material, and (D) covalent bonding of a material and heparin.”

“Of the above methods, the methods (2) and (3) are capable of affording a stable antithrombogenicity during a long-term contact with body fluids, since protein adsorbs onto the surface of a material to form a biomembrane-like surface. At the initial stage when the material has been introduced into the body (blood contact site) and when various coagulation factors etc. in the body have been activated, however, it is difficult to achieve sufficient antithrombogenicity without an anticoagulant therapy such as heparin administration.”

Other antithrombogenic methods and compositions are also well known. Thus, by way of further illustration, United States published patent application 20010016611 discloses an antithrombogenic composition comprising an ionic complex of ammonium salts and heparin or a heparin derivative, said ammonium salts each comprising four aliphatic alkyl groups bonded thereto, wherein an ammonium salt comprising four aliphatic alkyl groups having not less than 22 and not more than 26 carbon atoms in total is contained in an amount of not less than 5% and not more than 80% of the total ammonium salt by weight. The entire disclosure of this published patent application is hereby incorporated by reference into this specification.

Thus, e.g., U.S. Pat. 5,783,570 discloses an organic solvent-soluble mucopolysaccharide consisting of an ionic complex of at least one mucopolysaccharide (preferably heparin or heparin derivative) and a quaternary phosphonium, an antibacterial antithrombogenic composition comprising said organic solvent-soluble mucopolysaccharide and an antibacterial agent (preferably an inorganic antibacterial agent such as silver zeolite), and to a medical material comprising said organic solvent soluble mucopolysaccharide. The organic solvent-soluble mucopolysaccharide, and the antibacterial antithrombogenic composition and medical material containing same are said to easily impart antithrombogenicity and antibacterial property to a polymer to be a base material, which properties are maintained not only immediately after preparation of the material but also after long-term elution. The entire disclosrure of this United States patent is hereby incorporated by reference into this specification.

By way of further illustration, U.S. Pat. 5,049,393 discloses anti-thrombogenic compositions, methods for their production and products made therefrom. The anti-thrombogenic compositions comprise a powderized anti-thrombogenic material homogeneously present in a solidifiable matrix material. The anti-thrombogenic material is preferably carbon and more preferably graphite particles. The matrix material is a silicon polymer, a urethane polymer or an acrylic polymer. The entire disclosure of this United States patent is hereby incorporated by reference into this specification.

By way of yet further illustration, U.S. Pat. 5,013,717 discloses a leach resistant composition that includes a quaternary ammonium complex of heparin and a silicone. A method for applying a coating of the composition to a surface of a medical article is also disclosed in the patent. Medical articles having surfaces which are both lubricious and antithrombogenic, are produced in accordance with the method of the patent. The entire disclosure of this United States patent is hereby incorporated by reference into this specification.

Referring again to FIG. 2, and in the preferred embodiment depicted therein, a power supply (not shown in FIG. 2, but see element 102 of FIG. 1) provides electrical current to conductor/coil 117.

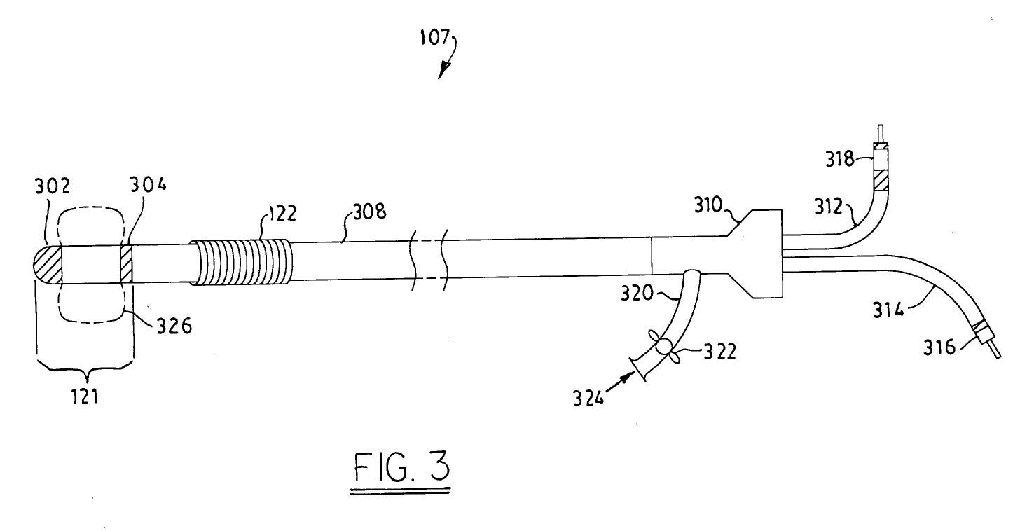

FIG. 3 is a schematic view of a catheter 107. Referring again to FIG. 3, and in the preferred embodiment depicted therein, it will be seen that combined distal pacing and cardioverter-defibrillator cathether 107 is comprised of a distal lead assembly 121 that is comprised of bipolar electrode set 302/304 and optionally air inflatable balloon 326 for catheter guidance during placement.

The catheter 107 also is comprised of distal cardioversion-defibrillation electrode 122 positioned on catheter 107 and attached to block 310. The block 310 is preferably adapted to introduce fluid, such as air. In the embodiment depicted, the block 310 is attached to lumens 312 and 314 which are fitted with pulse generator connectors 316 (e.g. DF-1connector ISO-11318) and 318 (e.g. IS-1 bipolar); and lumen 320, stopcock 322, and port 324. In one embodiment, not shown, catheter 107 contains a second lumen (not shown) for guiding the lead 107 into position over a conventional guide wire (not shown).

Referring again to FIG. 3, pacing electrodes 302 and 304, and cardioversion-defibrillation electrode 122 are internally connected to pacing electrode connector 318 and cardioverter-defibrillator electrode connector 316, respectively. Catheter 107 also preferably contains a minimum of one hollow lumen (not shown) attached to port 324 and optional balloon 326, which can be inflated to help fix the position of distal pacing electrodes 302 and 304.

FIG. 4 is a schematic of an adapter 400 which is adapted to provide an electrical connection from pulse generator 102 (in whatever configuration it may be in) to the assemblies 107, 109, and/or 114. Although one particular adapter 400 is illustrated in FIG. 4, many other such adapters may be used to effectuate the same function.

In the particular embodiment depicted in FIG. 4, adapter 400 is comprised of a space 402 in which a pulse generator 102 (not shown) may be disposed. In the embodiment depicted, the pulse generator (102) is disposed within a housing 405 that is comprised of a primary container 404 removably connected to removable latch. A controller 408 is connected to the pulse generator 102 (not shown) and also to output grounding connection 410 connected to container 404. The controller also connects the pulse generator 102 to ports 512, 514, 516, and 518, each of which is adapted to deliver energy and/or in information to one or more different locations.

In one embodiment, the adapter 400 is comprised of a housing (within which the pulse generator 102 may be disposed) and means for electrically isolating the pulse generator 102 from the biological organism. In one aspect of this embodiment, the housing provides both hermetic and electrical insulation from the biological organism.

Referring again to FIG. 1, and in one embodiment depicted therein, it will be seen that a contact clip 421 is connected to the interior wall 423 of primary container 404 and is removably engaged with the pulse generator 102 (not shown). In one preferred aspect this embodiment, the exterior of the pulse generator 102 is electrically connective, and such exterior makes electrical connection with the clip 421, which also preferably is electrically connective.

FIG. 6 is schematic illustration of yet another assembly 611 of the invention that is similar to the assembly 107 depicted in FIG. 3 but differs therefrom in that it also contains a secondary conductor 309 that has substantially the same location and the same functionality as conductors 117/118 of FIG. 1.

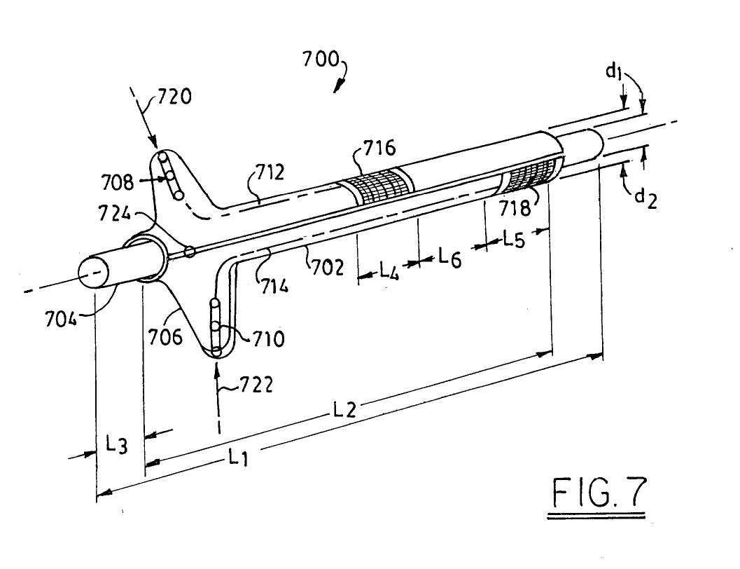

FIG. 7 is a schematic of another embodiment of a sheath 700. Referring to FIG. 7, it will be sent that tear-away sheath assembly 700 comprises a sheath 702 positioned on catheter 704, wherein catheter 704 is similar to pacing and cardioverter-defibrillator lead 107 shown in FIG. 3. Sheath 702 contains a proximally positioned flange 706 which contains connection ports 708 and 710 which are electrically connected through conductors 712 and 714 for making electrical contact with distally positioned cardioversion-defibrillation electrodes 716 and 718 which are positioned into contact with the tissues to be electrically stimulated (not shown in FIG. 7). Conductors 720 and 722 plug into connection ports 708 and 710 to connect lead 700 to the externally positioned pulse generator (not shown). Multiple perforations 724 pass along the length of sheath 702 and enable lead 700 to be translated along catheter 704 and split and removed from catheter 704 without disrupting contact between catheter 704 and external pulse generator (not shown).

In one embodiment, one or more perforations 724 are disposed on one or more sides and/or in one more locations of the sheath.

As will be apparent, the perforation(s) 724 preferably provide a weakened section of the sheath which, when subjected to force in, e.g., an axial direction, preferentially tears. Such a weakened section may be provided by aligning two or more perforations in sheath 724 in a straight line and/or a curve line. Alternatively, or additionally, such a weakened section may be provided by means of making a section of the sheath of thinner material than used elsewhere in the sheath, and/or of different material, and/or of material that preferentially decays and/or degrades with time.

Although a few preferred embodiments of the present invention have been shown and described, it will be appreciated by those skilled in the art that changes may be made in these embodiments without departing from the spirit and scope of the invention.

We claim:

1. An apparatus for the insertion of a temporary percutaneous cardioverter-defibrillator in contact with biological tissue, comprising:
(a) an sheath assembly comprises a sheath, wherein said sheath comprises an proximal section, a mid section, and a distal section, and wherein:
1. said proximal section is integrally connected to said mid section, and said mid section is integrally connected to said distal section,
2. a lumen extends substantially parallel to said sheath ftom said proximal section to said distal action,
(b) a conductor disposed on the periphery of said mid section of said sheath assembly,
(c) a catheter disposed within said lumen,
(d) means for flowing electrical current through said conductor and to said biological tissue, and
(e) means for flowing electrical current through said catheter and to said biological tissue.

2. The apparatus as recited in claim 1, further comprising a pulse generator.

3. The apparatus as recited in claim 2, wherein biological tissue is disposed within a biological organism.

4. The apparatus as recited in claim 3, wherein said pulse generator is disposed outside of said biological organism.

5. The apparatus as recited in claim 4, further comprising a defibrillator comprised of said pulse generator.

6. The apparatus as recited in claim 4, further comprising a pacemaker comprised of said pulse generator.

7. The apparatus as recited in claim 4, further comprising a cardioverter comprised of said pulse generator.

8. The apparatus as recited in claim 1, wherein said biological tissue is disposed within a biological organism.

9. The apparatus as recited in claim 8, further comprising a patch electrode selected from the group consisting of a cutaneous patch electrode and a subcutaneous patch electrode.

10. The apparatus as recited in claim 9, wherein said patch electrode is contiguous with an outside surface of said biological organism.

11. The apparatus as recited in claim 10, further comprising a pulse generator.

12. The apparatus as recited in claim 11, further comprising means for directing electrical energy to a location selected from the group consisting of said pulse generator, said patch electrode, said conductor disposed on said periphery of said mid-section of said sheath assembly, said catheter disposed within said lumen, and mixtures thereof.

13. The apparatus as recited in claim 12, comprising means for connecting said pulse generator with a device selected from the group consisting of said patch electrode, said conductor disposed on said periphery of said mid-section of said sheath assembly, said catheter disposed within said lumen, and mixtures thereof.

14. An assembly comprised of an apparatus for the insertion of a temporary percutaneous cardioverter-defibrillator in contact with biological tissue of a biological organism, wherein said assembly is comprised of a device comprising a proximal section, a mid section, and a distal section, wherein said proximal section is integrally connected to said mid section, and said mid section is integrally connected to said distal section, and wherein:
(a) said apparatus is comprised of a first conductor disposed on the periphery of said distal section of said assembly, wherein said conductor is disposed within the vascular system of said biological organism and in proximity to the heart of said biological organism,
(b)said apparatus is comprised of means for flowing electrical current through said conductor and to said biological tissue, and
(c) said assembly is comprised of an electrode connected to said apparatus, wherein said electrode is selected from the group consisting of a cutaneous electrode, a subcutaneous electrode, and mixtures thereof.

15. The assembly as recited in claim 14, wherein said apparatus further comprises a second conductor.

16. The apparatus as recited in claim 15, wherein said assembly is a sheath.

17. The apparatus as recited in claim 16, wherein said apparatus further comprises a lumen that extends substantially parallel to said sheath from said proximal section to said distal action,

18. The apparatus as recited in claim 17, wherein a cathether is disposed within said lumen.

19. The apparatus as recited in claim 18, further comprising means for flowing electrical current through said catheter to said biological tissue.

20. An assembly comprised of an apparatus for the insertion of a temporary percutaneous cardioverter-defibrillator in contact with biological tissue of a biological organism, wherein said assembly is comprised of a device comprising a proximal section, a mid section, and a distal section, wherein said proximal section is integrally connected to said mid section, and said mid section is integrally connected to said distal section, and wherein:
(a) said apparatus is comprised of a first conductor disposed on the periphery of said distal section of said device, wherein said conductor is disposed within the vascular system of said biological organism and in proximity to the heart of said biological organism,
(b)said apparatus is comprised of means for flowing electrical current through said conductor and to said biological tissue,
(c) said assembly is comprised of an electrode connected to said apparatus, wherein said electrode is selected from the group consisting of a cutaneous electrode and a subcutaneous electrode; and
(d) said apparatus is comprised of a second conductor diposed around said mid-section of said assembly.

21. An assembly comprised of an apparatus for the insertion of a temporary percutaneous cardioverter-defibrillator in contact with biological tissue of a biological organism, wherein said assembly is comprised of a device comprising a proximal section, a mid section, and a distal section, wherein said proximal section is integrally connected to said mid section, and said mid section is integrally connected to said distal section, and wherein:
(a) said apparatus is comprised of a first conductor disposed on the periphery of said distal section of said assembly, wherein said conductor is disposed within the vascular system of said biological organism and in proximity to the heart of said biological organism,
(b)said apparatus is comprised of means for flowing electrical current through said conductor and to said biological tissue,
(c) said assembly is comprised of an electrode connected to said apparatus, wherein said electrode is a cutaneous or subcutaneous electrode.
(d) said apparatus is comprised of a sheath disposed around said first conductor, and means for removing said sheath from said conductor by tearing at least one portion of said sheath.

22. The assembly as recited in claim 21, wherein said means for removing said sheath is comprised of weakened section of said sheath that preferentially tears upon the application of a tearing force.

23. The assembly as recited in claim 22, wherein said weakened section of said sheath is comprised of a multiplicity of perforations.

24. The assembly as recited in claim 23, wherein said multiplicity of perforations are arranged in a straight line.

25. The assembly as recited in claim 14, wherein said assembly further comprises a housing.

26. The apparatus as recited in claim 14, wherein said housing is comprised of a housing comprised of a pulse generator and means for electrically isolalating said pulse generator from said biological organism.

27. The apparatus as recited in claim 14, wherein said assembly further comprises a controller.

28. The apparatus as recited in claim 26, further comprising means for electrically connecting said pulse generator to said device.

29. The apparatus as recited in claim 14, further comprising a pulse generator, a patch electrode, and a connection between said pulse generator and said patch electrode.

30. The apparatus as recited in claim 29, further comprising means for checking the integrity of said connection between said pulse generator and said patch electrode.
